(19) United States
(12) Patent Application Publication
(10) Pub. No.: US 2007/0275032 A1
(43) Pub. Date: Nov. 29, 2007

(54) Use Of A Mixture For The Production Of An Agent For Treating Defective Or Degenerated Cartilage In The Production Of Natural Cartilage Replacement In Vitro

(75) Inventors: Markus Wimmer, Chicago, IL (US); Mauro Alini, Davos Platz (CH); Thomas Kaup, Davos Platz (CH)

Correspondence Address: JONES DAY, 222 EAST 41ST STREET, NEW YORK, NY, 10017-6702, US

(73) Assignee: Synthes (U.S.A.), West Chester, PA (US)

(21) Appl. No.: 10/591,833

(22) PCT Filed: Mar. 5, 2004
(86) PCT No.: PCT/CH04/00131

§ 371 Date: Jul. 16, 2007

(51) Int. Cl. A61K 38/16; A61K 31/728; A61P 19/02

(52) U.S. Cl. 424/423; 424/93.7; 514/120; 514/54; 514/8

(57) ABSTRACT

A mixture of one or several substances from group A) lubricin, proteoglycan 4 (PRG4) and phosphollipid (SAPL); with one or several substances from group B) hyaluronic acid, glycosaminoglycan and derivatives of said substances; dissolved in a solvent, used for the production of an agent for treating defective or degenerated cartilage in vivo. Said mixture can also be used to produce natural cartilage replacement in vitro.

### 1. FIELD OF THE INVENTION

The invention relates to a method for using a mixture of one or more substances of group A) lubricin, proteoglycan 4 (PRG4) and phospholipids (SAPL) with one or more substances of group B) hyaluronic acid, glycosaminoglycan and derivatives of these substances dissolved in a solvent, for the production of an agent for the treatment of defective or degenerated cartilage in vivo, as well as to the use of this mixture for the production of natural cartridge replacement in vitro.

### 2. BACKGROUND

Permanent pain, immobility and an impairment of the joint are typical indications of injury to the cartilage due to an accident or osteoarthrosis. The success of surgical interventions in joint injuries, such as osteotomy, transplantation of the perichondrium or the use of an arthroplastic material, is limited. As a rule, the natural hyalin structure of a healthy cartilage is never attained by surgery.

For treating cartilage defects, every effort is made to implant frameworks of polymer materials, which will be colonized with chondrocytes. These materials function here as carrier material for the chondrocytes and are available as absorbable or non-absorbable materials. In recent years, frameworks from natural and synthetic absorbable carrier materials were developed and tested. In so doing, it was noted that cartilage-like constructions, which had been raised in vitro, attained neither the biochemical nor the biomechanical properties of in vivo tissue.

Several methods are used for the clinical treatment of cartilage defects. In the past, the damaged cartilage tissue was predominately removed mechanically. Newer treatment methods transplant chondrocytes and periosteum or perichondrium for closing the lesion.

The method of milling out was described for the first time in 1959 by Pridie. The method of abrasive removal was developed in the 1980s. Both methods are based on the same principle. First, the defective cartilage sites are removed to the bleeding bone. Enough cartilage is removed so the transition from bone to cartilage is formed exclusively by undamaged cartilage. The healing of the cartilage is promoted by the rich supply of nutrients of the opened blood vessels of the bone. Numerous studies have shown that the regrown tissue consists predominantly of fiber cartilage and not of the hyaline cartilage, which is necessary for permanent regeneration.

Other methods make use of osteochondral transplants. As autograph or allograph, these transplants are inserted into the cartilage defect and anchored in the subchondral bone. In the first case (autograph), the organ donor and the host are one and the same person and, in the second case (allograph), they are different persons, but of the same species. Cylindrical cartilage studs, together with the subchondral bone, are removed from the donor region with the help of a stamping tool and anchored in the defect zone by means of a prefabricated press fit. One or more studs (→mosaic plastic), depending on the size of the defect zone, are used to close the damaged surface.

For transplanting chondrocytes, the latter are removed from cartilage regions of the knee, which are not stressed as much. The cells removed are propagated for 14 to 21 days in nutrient solution. After they have been cultured, the cells are injected into the region of the defect and covered with a piece of periosteum or perichondrium. After 2 years, it can be shown by a biopsy that hyaline cartilage has formed. In one study, the clinical result of 14 of 16 patients was described as good to very good. A study in Sweden with 400 patients showed comparable results.

The function of cartilage in joints consists of, on the one hand, absorbing and distributing forces, which arise when the joint is stressed, and, on the other hand, making available a lubricating surface, which prevents the abrasion and degradation of the joint. The first function is ensured by a unique composition and structure of the extracellular matrix, whereas the second function depends on a functional cartilage-synovial fluid interface. There is interference with these functions especially in patients with cartilage surfaces, which are degeneratively changed or otherwise affected.

The invention is to provide a remedy here. It is an object of the invention, on the one hand, to provide an agent for the treatment of defective or degenerated cartilage in vivo and, on the other hand, to make available an improved production of natural cartilage replacement in vitro, especially for cartilage defects in the joint region.

### 3. SUMMARY OF THE INVENTION

Pursuant to the invention, this objective is accomplished with an agent, which has the distinguishing features of claim 1, as well as with a use of this agent, which has the distinguishing features of claim 9.

As lubricin, the lubricating glycoprotein-1 (LGP-1) is named, which is produced from the same gene as the megakaryocyte stimulating factor (MSF) by alternative splicing. Lubricin has a molecular weight of approximately 230 kDa (purified form in human synovial fluid) and is glycosylated to a high degree.

As proteoglycan 4 (PRG4), the surface zone protein (SZP) is named, which is obtained by alternative splicing from the MSF gene. It has a molecular weight of approximately 340 kDa (from human joint cartilage) and carries several oligosaccharides groups, as well as glycosaminoglycan chains. It has turned out that the use of SZP and similar substances (group A) in the inventive mixture not only has a strong lubricating effect, but also acts as a chondro-protective molecule, which gives protection for the lower-lying cartilage cells.

Originally, SZP was isolated and purified from culture liquids from explants, which originated from the surface zone of bovine cartilage. SZP can be synthesized by chondrocytes in the surface zone, but not by those from the middle and lower zones.

Hyaluronic acid consists of glucouronic acid and acetylglucosamine, which build up the disaccharides, hyalubironic acid. As a result of its filamentous, unbranched molecular structure, hyaluronic acid forms highly viscous solutions. Admittedly, hyaluronic acid does not have any direct lubricating properties. However, it is important for the rheological behavior of the synovial fluid by adjusting the viscosity suitably. Such an adjustment prevents the synovial fluid flowing out during the loading phase of the joint.

Surprisingly, it was found that the mixing of lubricin (or similar substances of group A) with a hyaluronic acid (or similar substances of group B) in a suitable solvent reinforces the action of these two substances in a synergistic manner.

Further advantageous developments of the invention are characterized in the dependent claims.

### 4. DETAILED DESCRIPTION OF THE INVENTION

The advantages, attained by the invention, are, essentially, the following:
In patients with osteoarthrosis, the improved lubrication results in a reduction in pain and/or delay or even complete prevention of further degradation of the cartilage.
In patients with hemiarathroplasty, the improved lubrication results in a reduction in the cartilage degeneration and/or an improved abrasion of the artificial joint. As a result, the service life of the implant increases and a reversion can be prevented or delayed.
In patients with cartilage trauma or surgical interventions, the improved lubrication results in a reduction in the shear forces at the wound. As a result, there is better healing of the two halves of the tissue.
The lubrication of joints in the case of osteoarthrosis, hemiprostheses, after an osteochondral transplant and autologous cell transplant (ACT) or after a meniscus operation.

The improved lubrication is achieved with natural joints (especially in cases of osteoarthritis and rheumatoid arthritis) as well as with artificial joints. In cases of a total of hip prosthesis, the lubrication between the polyethylene of the acetabulum component and the metal of the hip head of the shaft component is improved.

In certain embodiments, the phospholipids used are surface active in nature. The interfacial lubrication, resulting therefrom, is responsible for less cartilage damage in the further course.

In certain embodiments, the hyaluronic acid used has a molecular weight of at least 1×10⁶ Da.

In certain embodiments, the ratio by weight of the substances of group A (lubricin, proteoglycan 4 (PRG4) and phospholipids (SAPL)) to the substances of group B (hyaluronic acid, glycosaminoglycan and derivatives of these substances) ranges from 0.05 to 0.40, and preferably from 0.08 to 0.25.

In certain embodiments, the solvent used is a Ringer solution, preferably a physiological salt solution.

In certain embodiments, the concentration of the substances of group A in the solvent preferably ranges from 0.02 to 0.05% by weight, and the concentration of the substances of group B preferably ranges from 0.2 to 0.4% by weight.

The mixture of one or more substances of group A (lubricin, proteoglycan 4 (PRG4) and phospholipids (SAPL)) with one or more substances of group B (hyaluronic acid, glycosaminoglycan and derivatives of these substances) dissolved in a solvent can also be used for the production of natural cartilage replacement in vitro. Such a mixture can also be used for a method of producing a cartilage replacement material for cartilage defects in the joint region, wherein said cartilage replacement material comprises an open-pored, elastic cell-carrier body being populated in its pores with chondrocytes and the mixture, dissolved in a physiologically acceptable solvent, is being brought into contact with the chondrocytes.

For this method, the solvent is preferably moved with a lamina flow over the cell-carrier body.

In the case of a particular embodiment of this inventive method, an axial force and a rotational force are applied on the cell-carrier body simultaneously with a ball joint-like device. Preferably, the rotation of the ball joint-like device is carried out about two axes, which are orthogonal to one another. The advantage of this measure is based therein that, with an appropriate phase shift, movement trajectories can be set, which come close to those of human joints with respect to distance, form and speed.

The invention and further developments of the invention are explained in even greater detail in the following by means of several examples.

### 5. EXAMPLES

### 5.1. Example 1

Lubricin (4 mg) and 40 mg of hyaluronic acid were dissolved in 20 mL of physiological salt solution (Ringer solution). Over a period of 10 weeks, 2 mL of the solution, so obtained, were injected in situ once a week into the knee joint of a patient with osteoarthritis. Before the injection, the joint was aspirated, in order to prevent dilution of the solution injected.

The patient treated therewith had less pain and improved mobility of the knee joint. A further flushing at a later time showed a distinct reduction in loose cartilage particles in the aspirate.

### 5.2. Example 2

Lubricin (4 mg) and 40 mg of glycosaminoglycan were dissolved in 20 mL of physiological salt solution (Ringer solution). For a period of 10 weeks, 1 mL of the solution, so obtained, was injected in situ once a week into the hip joint of a patient with osteoarthritis. Before the injection, the joint was aspirated in order to prevent dilution of the solution injected. The patient treated therewith had less pain and improved mobility of the hip joint.

### 5.3. Example 3

Lubricin (5 mg) and 40 mg of hyaluronic acid were dissolved in 20 mL of physiological salt solution (Ringer solution). For a period of 5 weeks, 2 mL of the solution, so obtained, was injected in situ once a week into the finger joints of a patient with rheumatoid arthritis. Before the injection, the joint was aspirated in order to prevent dilution of the solution injected. The patient treated therewith had less pain and a better function of the hand due to the increased extent of movement of the finger joints.

### 5.4. Example 4

After an osteochondral transplantation, a solution of 6 mg lubricin and 45 mg hyaluronic acid was injected into the closed joint capsule of a patient. The solvent consisted of 25 mL of physiological salt solution (Ringer solution), into which 5% of human serum of the same patient had been mixed. The endoscopic examination after the physiotherapeutic therapy of the joint showed improved healing of the sections between the host and donor tissue. The patient was free of pain and could undertake his usual activities.

### 5.5. Example 5

Chondrocytes were isolated from the region of the surface of the knee joint, which carried no weight and had a defect, and implanted directly into an open-pored elastic cell-carrier body. The cell-carrier body consisted of a cylindrical, porous, biodegradable polyurethane framework having a size of 8 mm×4 mm, corresponding to that of the defect. The cell density was 25-30×10⁶. The cell-carrier body, the pores of which were populated with chondrocytes, was cultured in “Dulbecco's modified Eagles medium” (DMEM), to which 5% of human serum (of the same patient), a number of nonessential amino acids, namely 1-alanine (0.89 mg/L), 1-asparagine (1.32 mg/L), 1-aspartic acid (1.33 mg/L), 1-glutamic acid (1.47 mg/L), glycine (0.75 mg/L), 1-proline (1.15 mg/L) and 1-serine (1.05 mg/L), as well as 40 μg/L 1-proline had been added.

Two days after the cell-carrier body had been populated, 50 μg/mL of ascorbic acid were added. In addition, immediately before the start of the mechanical stress, 0.2 mg of lubricin and 2 mg of hyaluronan per milliliter of medium where added, of which 3 mL were required. The medium was exchanged daily. After a six-day cell culture, the cell-carrier body was subjected to the mechanical stress as described below.

The mechanical stressing of the cell-carrier body took place in a so-called bioreactors system, in which the cell-carrier body was subjected to the action of a ball, so that rotational as well as axial forces could be exerted on the cell-carrier body. Twice a day, a one-hour mechanical stress of this type was exerted on the cell-carrier body. In one series of experiments, this procedure was carried out from 3 days after 28 days.

The aforementioned addition of 0.2 mg of lubricin and 2 mg of hyaluronan resulted in an improved production of functional cartilage-like tissue, which, after implantation in a cartilage defect, showed an improved physiological effect and led to optimum healing of the cartilage defect.

### 6. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

1. A method for treating defective or degenerated cartilage in vivo, comprising administering to a subject a mixture of (i) one or more substances of group A selected from the group consisting of lubricin, proteoglycan 4 (PRG4) and phospholipids (SAPL); and (ii) one or more substances of group B selected from the group consisting of hyaluronic acid, glycosaminoglycan and derivatives of these substances, wherein said substances are dissolved in a solvent.

2. The method of claim 1, wherein said phospholipids are surface active in nature.

3. The method of claim 1, wherein said hyaluronic acid has a molecular weight of at least 1×10⁶ Da.

4. The method of claim 1, wherein the ratio by weight of the substances of group A to the substances of group B ranges from 0.05 to 0.40.

5. The method of claim 1, wherein the ratio by weight of the substances of group A to the substances of group B ranges from 0.08 to 0.25.

6. The method of claim 1, wherein said solvent is a Ringer solution or a physiological salt solution.

7. The method of claim 1, wherein the concentration of the substances of group A dissolved in the solvent ranges from 0.02 to 0.05% by weight.

8. The method of claim 1, wherein the concentration of the substances of group B dissolved in the solvent ranges from 0.2 to 0.4% by weight.

9. A method for the production of a natural cartilage replacement material, comprising dissolving in a solvent a mixture of (i) one or more substances of group A selected from the group consisting of lubricin, proteoglycan 4 (PRG4) and phospholipids (SAPL); and (ii) one or more substances of group A selected from the group consisting of hyaluronic acid, glycosaminoglycan and derivatives of these substances.

10. The method of claim 9, wherein said natural cartilage replacement material comprises an open-pored, elastic cell-carrier body populated in its pores with chondrocytes, and wherein said mixture, dissolved in a physiologically acceptable solvent, is brought into contact with the chondrocytes.

11. The method of claim 10, wherein said solvent is moved over the cell-carrier body with a laminar flow.

12. The method of claim 10 or 11, wherein by means of a joint-like device, an axial and a rotational force is exerted simultaneously on the cell-carrier body.

13. The method of claim 12, wherein the rotational force is carried out about two axes, which are orthogonal to one another.

14. The method of claim 1, wherein the mixture comprises lubricin and hyaluronic acid.

15. The method of claim 9, wherein the mixture comprises lubricin and hyaluronic acid.

16. The method of claim 1, wherein the mixture comprises lubricin and hyaluronic acid.

17. The method of claim 9, wherein the mixture comprises lubricin and glycosaminoglycan.
